(19) United States
(12) Patent Application Publication
(10) Pub. No.: US 2017/0112656 A1
(43) Pub. Date: Apr. 27, 2017

(54) Medical Hand and Arm Protection Apparatus and Method of Protection

(71) Applicants: Fereshteh K. Maynard, Conway, AR (US); Jeffery B. Wilkerson, Monticello, AR (US); Victoria L. Eskew, Benton, AR (US)

(72) Inventors: Fereshteh K. Maynard, Conway, AR (US); Jeffery B. Wilkerson, Monticello, AR (US); Victoria L. Eskew, Benton, AR (US)

(21) Appl. No.: 15/397,060

(22) Filed: Jan. 3, 2017

Related U.S. Application Data
(63) Continuation-in-part of application No. 14/951,791, filed on Nov. 25, 2015, now Pat. No. 9,566,396.
Application No. 14/951,791 is a continuation of application No. 14/146,532, filed on Jan. 2, 2014, now Pat. No. 9,227,026.
Application No. 14/146,532 is a continuation of application No. 13/007,304, filed on Jan. 14, 2011, now Pat. No. 8,646,457.

(51) Int. Cl. A61F 5/37; A61G 13/10; A61M 5/158; A61G 13/12

(52) U.S. Cl. CPC A61F 5/3769; A61G 13/124; A61G 13/1235; A61G 13/101; A61M 5/158

(57) ABSTRACT

A medical hand and arm protection apparatus including a protective shell capable of receiving a patient's hand and arm. Connected to the protective shell is an attachment device to anchor the hand and arm protector in place, such as a clamp, slip cover or strap. The protective shell also has one or more flaps that allow the medical professional access to the interior of the protective shell to view the patient's IV sites and other medical equipment attached to the patient's hand or arm while the protective shell is anchored in place.

### CROSS-REFERENCE TO RELATED APPLICATIONS

This application is a continuation-in-part application of, and claims the benefit of, U.S. patent application Ser. No. 14/951,791, filed Nov. 25, 2015, which is a continuation application of, and claims the benefit of, U.S. patent application Ser. No. 14/146,532, filed Jan. 2, 2014, now U.S. Pat. No. 9,227,026 and issued on Jan. 5, 2016, which is a continuation application of, and claims the benefit of, U.S. patent application Ser. No. 13/007,304, filed Jan. 14, 2011, now U.S. Pat. No. 8,646,457 and issued on Feb. 11, 2014, which are all incorporated by reference in their entirety.

### STATEMENT REGARDING FEDERALLY FUNDED SPONSORED RESEARCH OR DEVELOPMENT

Not applicable

### BACKGROUND OF THE INVENTION

1. Field of the Invention

The present invention relates to medical hand and arm protectors, and in particular to medical hand and arm protectors capable of allowing access to intravenous (IV) sites, and other medical equipment connected to the patient's hand and arm during surgery, medical treatment, and/or recovery.

2. Brief Description of the Related Art

During many types of surgeries and medical procedures, including neurosurgery, cardiac surgery, ear, nose, and throat (ENT) surgery, robotic-performed surgeries, pacemaker procedures, I-port procedures, and other local procedures, the arms of the patient must be tucked to their side. Most commonly, the patient's arm is placed on a piece of foam either on the bed or a sled and taped to another piece of foam that is placed on top of the patient's arm. The draw sheet that the patient is laying on is then pulled over the arm and tucked back underneath the patient to secure their arm.

The inventors have observed that this process of tucking the patient's arms commonly requires up to 15 minutes of valuable time in the operating room. In addition, the inventors have recognized the potential for nerve injuries to the patient's arm, burns to the patient's hands, and the lack of easy access to IV sites using the prior art apparatus and method.

Nerve damage can result either because the arm is pulled too tight, the arm is not resting on a sufficiently padded surface, or because the arm is not positioned anatomically correct during the tucking process. Ulcers from decreased blood circulation can also result when the pressure points of the arm or bony prominences are not padded properly. In addition to during surgery and other medical procedures, nerve damage can also occur in patients that are bedridden, especially when the patients are unable to move their arms.

The inventors also recognize the risk for burns to the patient's hands during surgery. If the patient's finger tips are not properly covered during surgery, the fingers may come in contact with a hot surface, including the bed, the sled, or a cautery that is being used during the surgery.

As described above and in U.S. Pat. No. 5,546,963 to Doody, surgical arm protectors are known in the art. However, the prior art arm protectors do not allow adequate access to the patient's IV sites, and other medical instruments connected to the patient's arm or hand during surgery, medical treatment, and/or recovery. Specifically, all patients have at least one IV connected to their hand or arm ranging from the antecubital region of the arm, forearm, wrist, and/or back of the hand. In addition, in some cases, patients require an arterial line which is usually placed in the wrist to draw blood to monitor the patient's blood pressure and blood oxygen level. In order to draw blood from this line, the anesthesiologist must have free access to this line. However, with the prior art apparatus and method of tucking, the anesthesiologist is frequently required to get under the sheet to reposition and re-tuck the arm because the arterial line is extremely sensitive. The pressure from tight tucking causes movement and sometimes loss of the line. In addition, the arterial line and the other IV lines can get caught in the frame of the bed and the other appliances clamped to the bed during surgery if they are not properly stowed and secured. Easy IV site access is even more challenging in robotic surgeries where the patient is placed in a sharp trendelenberg position.

It would therefore be desirable to develop a medical hand and arm protector that is capable of providing support and protection of the arm during surgery, medical treatment, and/or recovery, while also allowing the medical professional access to IV sites and other medical equipment that is connected to the patient's hand and arm.

### BRIEF SUMMARY OF THE INVENTION

The present invention is directed to a medical hand and arm protection apparatus including a protective shell capable of receiving a patient's hand and arm. Connected to the protective shell is an attachment device to anchor the hand and arm protector in place, such as a clamp, slip cover, or supporting strap. The protective shell also has one or more flaps that allow the medical professional access to the interior of the protective shell to view the patient's IV sites and other medical equipment attached to the patient's hand or arm while the protective shell is anchored in place.

These and other features, objects and advantages of the present invention will become better understood from consideration of the following detailed description of the preferred embodiments, in conjunction with the drawings as described immediately below.

### BRIEF DESCRIPTION OF THE SEVERAL VIEWS OF THE DRAWINGS

FIG. 1 is a perspective view of the first side of the first preferred embodiment of the medical hand and arm protection apparatus.

FIG. 2 is a perspective view of the second side of the first preferred embodiment of the medical hand and arm protection apparatus.

FIG. 3 is a perspective view of the first side of the first preferred embodiment of the medical hand and arm protection apparatus showing the releasable flaps formed when the perforations are broken.

FIG. 4 is a top view of the second end of the first preferred embodiment of the medical hand and arm protection apparatus.

FIG. 5 is a top view of the second end of the second preferred embodiment of the medical hand and arm protection apparatus.

FIG. 6 is a perspective view of the first side of the second preferred embodiment of the medical hand and arm protection apparatus.

FIG. 7 is a perspective view of the third preferred embodiment of the medical hand and arm protection apparatus.

FIG. 8 is a top view of the fourth preferred embodiment of the medical hand and arm protection apparatus with a positioning device.

FIG. 9 is a perspective view of the fifth preferred embodiment of the medical hand and arm protection apparatus with a surgical sled.

FIG. 10 is a perspective view of the sixth preferred embodiment of the medical hand and arm protection apparatus with a surgical sled.

FIG. 11 is a perspective view of the seventh preferred embodiment of the medical hand and arm protection apparatus.

### DETAILED DESCRIPTION OF THE INVENTION

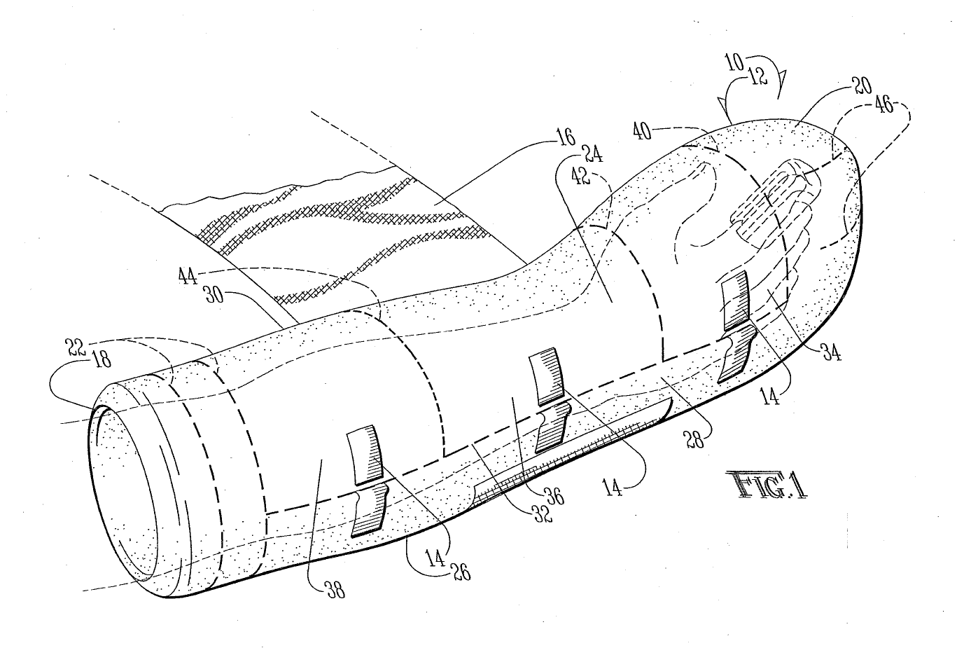

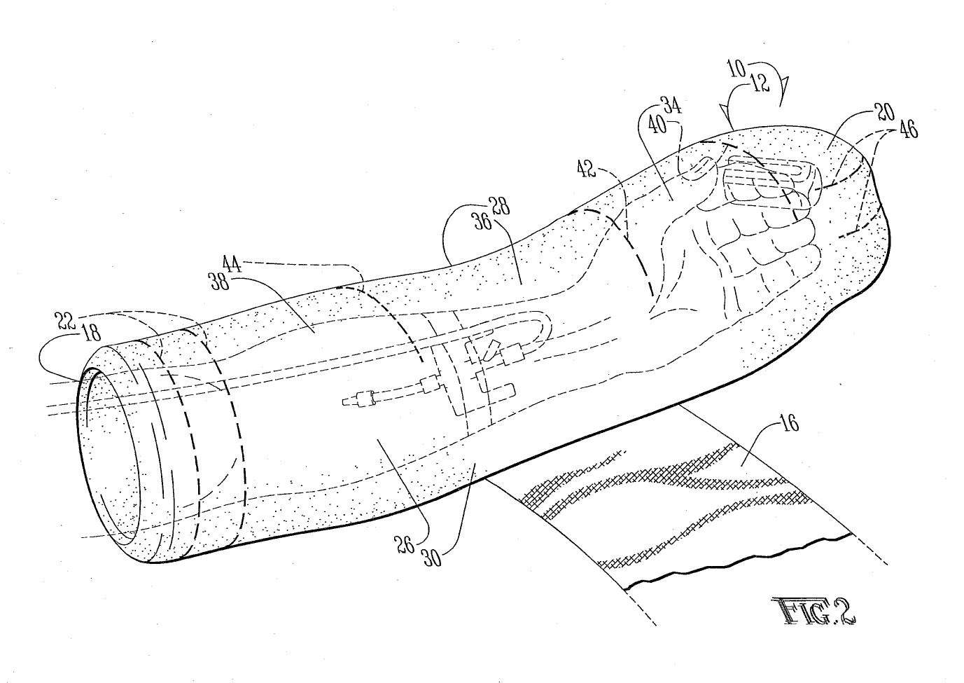

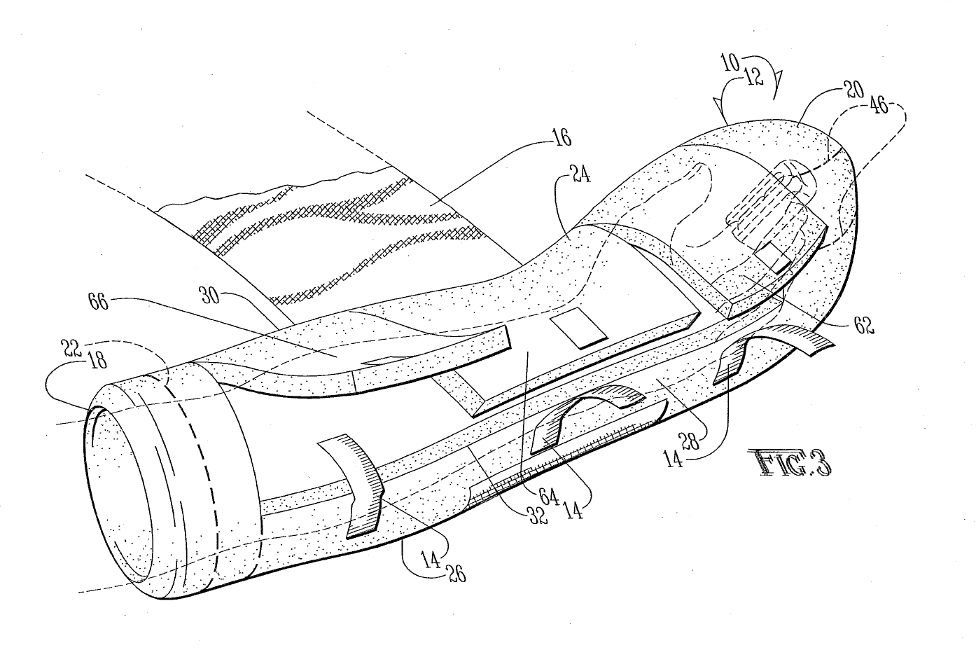

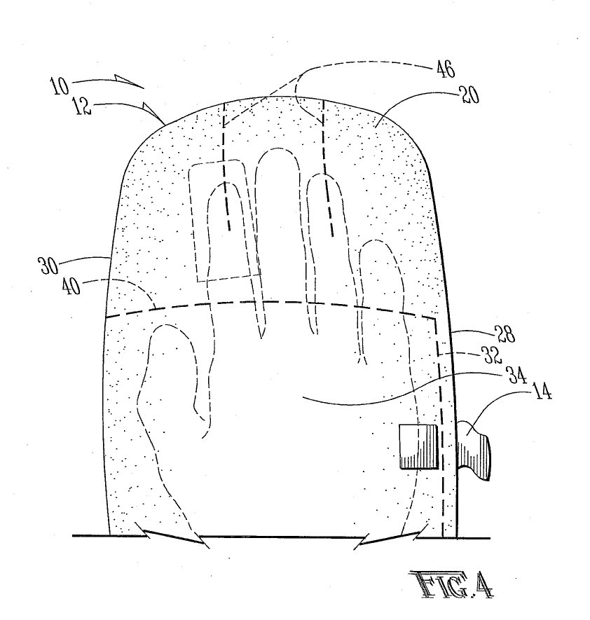

With reference to FIGS. 1-11, the preferred embodiments of the present invention may be described. The medical hand and arm protection apparatus 10 is comprised of a protective shell 12 and connecting strap(s) 14. The protector 10 is universal for both right and left arms of patients. In some embodiments (described below), the apparatus 10 includes a sheet 16.

In the preferred embodiments, the protective shell 12 has a first end 18 and a second end 20. In some embodiments, the shell 12 is open at the first end 18 such that a hand and arm can be slid into the shell 12, and closed at the second end 20 to fully cover and protect the hand and fingers of the patient. The hand and arm can be slid into the shell 12 either flat (i.e. top of hand facing up and palm of hand facing down) or on their side. The protective shell 12 is preferably made of foam or other material that is light in weight, soft, inexpensive, and capable of being manipulated. The shell 12 is preferably cylindrical in shape, but it may be rectangular or other suitable shapes. The shell 12 is preferably 27 inches in length. However, for child patients, the shell is preferably shorter in length. The interior of the protective shell 12 is large enough to fully engage and enclose the patient's hand and arm, and the medical equipment that is attached to the patient's hand or arm, including but not limited to, IV lines, pulse oximeters, and massaging arm wrap devices.

In some embodiments, the protective shell 12 is perforated. A perforation is defined as a line of small holes for tearing. Perforation 22 is located at the first end 18 of shell 12 and extends completely around the circumference of the shell 12. Perforation 22 can be broken which allows the shell 12 to be shortened to adapt it to the length of the patient's arm. In the preferred embodiments, the shell 12 has at least one perforation 22 preferably 1.5 inches from the first end of the shell 12. The shell may have an additional perforation 22 which is 1.5 inches from the first perforation. Thus, in the preferred embodiment, the shell is 27 inches in length and, through use of the perforations 22, can be quickly adapted to 25.5 inches or 24 inches in length. The number of perforations 22 and the distance between perforations 22 are variable.

In the first preferred embodiment, the protective shell 12 has a top section 24 and a bottom section 26, and a first side 28 and a second side 30. Along the first side 28 of the protective shell 12 is perforation 32. Perforation 32 extends longitudinally along the entire first side 28 of the shell 12. Perforation 32 preferably runs along the middle of the first side 28 of the shell 12.

The top section 24 of shell 12 is the top cross-section of the shell 12. Conversely, the bottom section 26 of shell 12 is the bottom cross-section of the shell 12. The top section 24 of protective shell 12 has perforated panels 34, 36, 38 which allow the medical professional access to the hand and arm. In the preferred embodiment, perforation 32 is laterally intersected by three perforations 40, 42, 44, which create perforated panels 34, 36, 38. Perforations 40, 42, 44 extend laterally along the entire top section 24 of the shell 12. The first perforated panel 34 is in the hand region of the surgical arm protector 10. The second perforated panel 36 is in the wrist region and the third perforated panel 38 is in the elbow region. The elbow region includes the brachial, antecubital, and forearm sections of the arm. Thus, for example, if the medical professional needs to check the IV site located in the patient's wrist, perforated panel 36 is utilized by breaking the perforations of longitudinally extending perforation 32 and laterally extending perforations 42, 44. Breaking the perforations of perforated panels 34, 36, 38 forms releasable flaps 62, 64, 66.

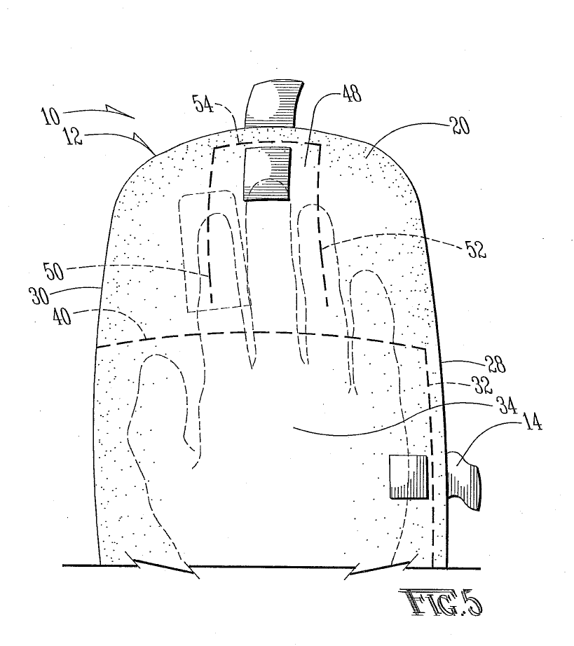

The second end 20 of the protective shell 12 also has one or more perforations 46 which extend longitudinally along the second end 20. Through use of the perforations 46, the medical professional can check the medical equipment attached to the patient's fingers, such as a pulse oximeter. The perforations 46 allow the medical professional to break the perforation 46 and use their fingers to pull the foam on either side of the broken perforation apart to view the interior of the second end 20 of the protective shell 12. In an alternative preferred embodiment, as shown in FIG. 5, the second end 20 of protective shell 12 has one or more perforated panels 48 created by two longitudinally extending perforations 50, 52 intersected by one laterally extending perforation 54.

On the first side 28 of the protective shell 12 is one or more connecting straps 14. In the preferred embodiment, the connecting strap 14 is a VELCRO® connector where one piece of the VELCRO® connector is attached to the perforated panels 34, 36, 38 (releasable flaps 62, 64, 66 when the perforations are broken) of the shell 12 and a complementary piece of the VELCRO® connector is attached to the bottom section 26 of the shell 12, directly below perforated panels 34, 36, 38. With the connecting strap 14, the medical professional can check the patient's IV or other medical equipment connections by breaking the perforations of the perforated panels 34, 36, 38, thus forming releasable flaps 62, 64, 66. The releasable flap(s) can then be secured by the connecting strap(s) 14. The connecting strap 14 need not be a VELCRO® connector, but instead can be any other means for connection as known to those skilled in the art, including hook and loop fasteners. In the alternative preferred embodiment where there are perforated panels in the second end of the protective shell 12, connecting straps 14 may also be utilized.

Attached to the first side 28 of protective shell 12 is a sheet 16. The sheet 16 is attached to the protective shell 12 by means well-known to those skilled in the art, including but not limited to, gluing and stitching. The sheet 16 is preferably attached directly below perforated panel 36. The sheet 16 is preferably 16 inches in length and 16 inches in width, but it may vary in size. The sheet 16 is made of a durable, nonabrasive, nonflammable, impermeable material that is capable of withstanding tearing and puncturing, such as the material used for medical gowns. Once the patient's hand and arm is inserted into the surgical hand and arm protector 10, the sheet 16 is tucked underneath the patient. As a result, the body weight of the patient anchors the protector 10 to the patient's side. When both the patient's arms and hands are in hand and arm protectors 10, the two sheets 16 may be connected underneath the patient to further secure the arms to the side of the patient. The means for connecting the two sheets 16 is of the type that is well-known to those skilled in the art.

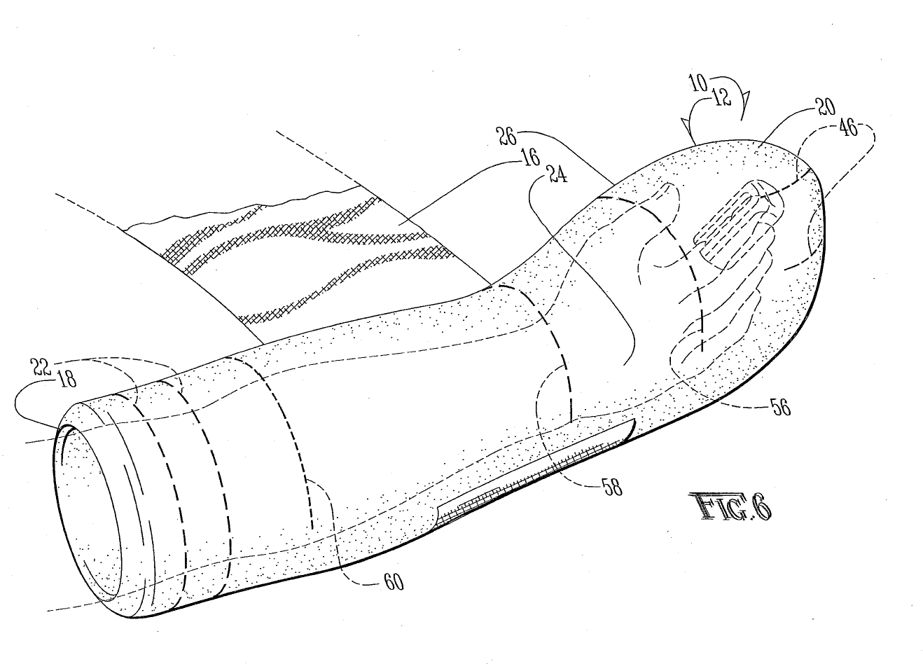

In a second preferred embodiment, as shown in FIG. 6, the protective shell 12 has a top section 24 and a bottom section 26. The top section 24 is the top cross-section of the shell 12, while the bottom section 26 is the bottom cross-section of the shell 12. Instead of having perforated panels 34, 36, 38 in top section 24 as in the first preferred embodiment, the top section 24 preferably has perforations 56, 58, 60. Perforations 56, 58, 60 extend laterally across the entire top section 24 of shell 12. Perforations 56, 58, 60 allow the medical professional to break the perforation and use their fingers to pull the foam on either side of the broken perforation apart to view the interior of the protective shell 12. Perforation 56 is in the hand region of the surgical arm protector 10. Perforation 58 is in the wrist region and perforation 60 is in the forearm and elbow region. While three laterally extending perforations is preferred, the number of perforations and their location can vary. In both the first and second preferred embodiments, each component of the hand and arm protection apparatus is preferably biodegradable.

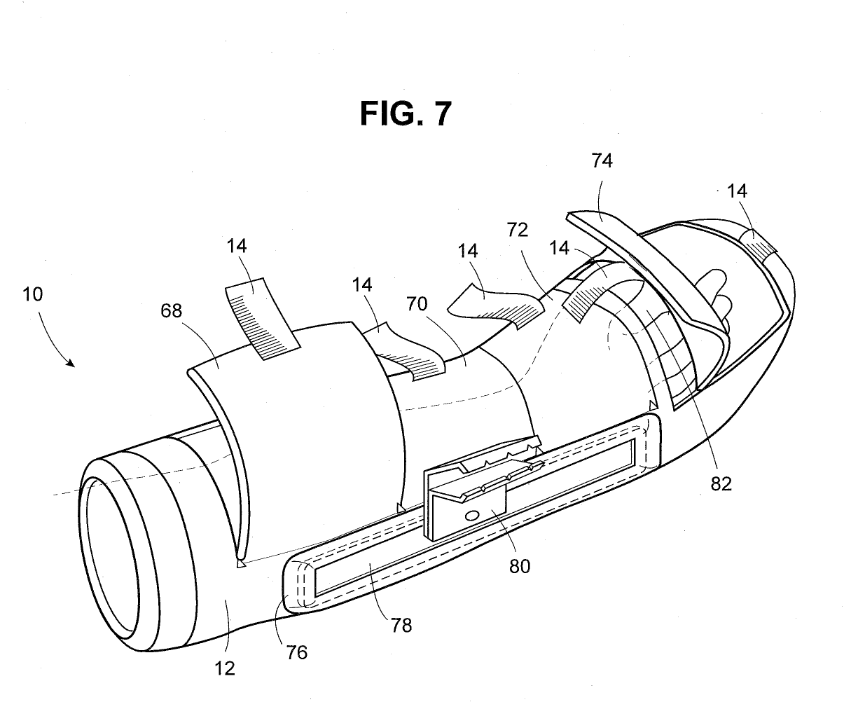

In a third preferred embodiment, as shown in FIG. 7, the top section 24 of the protective shell 12 include three flaps 68, 70, 72 positioned side-by-side. When the side flaps 68, 70, 72 are not open to view the patient's hand or arm, they are closed or secured by connecting straps 14. The connecting strap 14 preferably is a VELCRO® connector where one piece of the VELCRO® connector is attached to the side flaps 68, 70, 72 and complementary pieces of the VELCRO® connector are attached to the shell directly below the side flaps 68, 70, 72. The side flaps 68, 70, 72 are preferably in the hand region, wrist region, and elbow region of the patient. The second end 20 (i.e. closed end) of the shell 12 also includes an end flap 74. The end flap preferably is in the finger region of the patient. Like the side flaps, when the end flap is not open to view the patient's hand or arm, it is secured or closed by a connecting strap 14, preferably a VELCRO® connector. Directly behind the end flap (towards the first end 18 or open end of the shell 12) is a window opening 82 that extends across the top section 24 of the shell 12. Through the window opening 82, the medical professional is capable of observing the hand region of the patient at all times. In this embodiment, the apparatus 10 is reusable. The outer surface of the foam shell 12 preferably includes a vinyl layer that is easily wipeable to sanitize the apparatus 10 between uses.

The side of the shell 12 in this embodiment includes a pocket 76 to receive a support plate 78. The pocket 76 and support plate 78 are positioned on the shell 12 on the side closest to the medical bed. The support plate 78 is preferably metal, but it may alternatively be made of other durable materials that would be well-known to those skilled in the art. Connected to the support plate 78 is a clamp 80. For example, the clamp 80 may be connected to the support plate 78 with fasteners (e.g. screws) or via magnetic connection. The clamp 80 may be connected to the support plate 78 at different locations along the length of the support plate 78. The clamp 80 is used to secure the medical hand and arm protection apparatus 10 to the side of the medical bed or table 92. In this embodiment, the clamp 80 serves the purpose of the sheet 16 in securing the apparatus 10 in the proper position and thus the sheet 16 is not necessary. The clamp preferably includes a top and bottom piece that are capable of grasping a rail or other part of a medical bed or table, however, any clamp that would be well-known to those skilled in the art that is capable of securing the apparatus 10 to a medical bed or table 92 could alternatively be used.

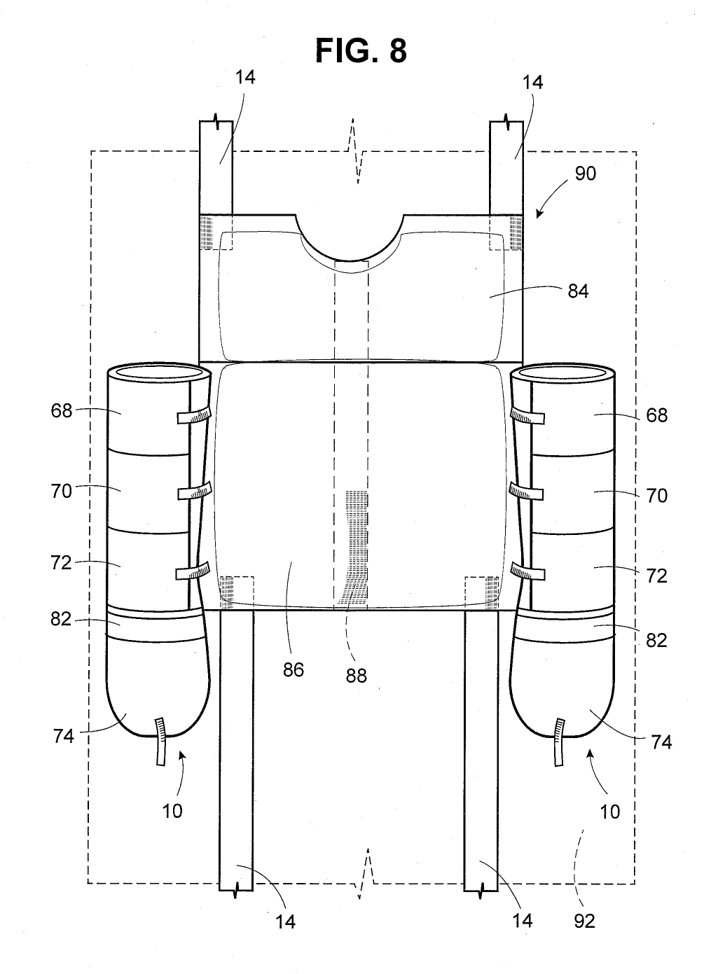

In the fourth preferred embodiment, as shown in FIG. 8, two medical hand and arm protection apparatuses 10 are attached to the left and right sides of a positioning device 90. The positioning device 90 is used to properly position and support a patient during surgery. The medical hand and arm protection apparatuses 10 are preferably of the type described in the third embodiment (minus the pocket 76, support plate 78, and clamp 80), but it may alternatively be of any other embodiment of the apparatus 10 described herein. The positioning device 90 includes a top portion 84 and bottom portion 86. When a patient is lying on the positioning device 90, the shoulder blades of the patient are preferably positioned at the bottom of the top portion 84 and the top of the patient's shoulders are positioned towards the top of the top portion 84 of the positioning device 90. The top portion 84 of the positioning device 90 preferably is a vacuum pack mattress or made up of bean bag material and includes an air release valve (not shown) for removing air from the top portion 84. The air is released from the top portion 84 after the patient is positioned on the top portion. Thus, when the air is released, the top portion 84 molds closely to the shape of the patient lying on the top portion 84 of the positioning device 90.

The bottom portion 86 of the positioning device 90 preferably is made up of foam mattress or similar soft material. When a patient is lying on the positioning device 90, the area of the patient's back just below the shoulder blades are positioned towards the top of the bottom portion 86 of the positioning device and the patient's pelvic region is positioned towards the bottom of the bottom portion 86 of the positioning device 86. The positioning device is secured to the medical bed 92 during surgery through use of connecting straps 14. Two connecting straps 14 preferably are attached to the top corners of the top portion 84 of the positioning device 90 and two connecting straps 14 are also preferably attached to the bottom corners of the bottom portion 86 of the positioning device. All four connecting straps extend underneath the medical bed or table 92. The two connecting straps 14 on the right side of the positioning device (one at the top and one at the bottom of the positioning device) are complementary to one another and are connected to one another on the underside of the medical bed or table 92. Similarly, the two connecting straps 14 on the left side of the positioning device (one at the top and one at the bottom of the positioning device) are complementary to one another and are connected to one another on the underside of the medical bed or table 92. The underside of the positioning device 90 includes a connecting strap 88 that is complementary to a connecting strap that extends down the middle of the medical bed or table 92. The connecting strap 88 of the positioning device connects to the connecting strap attached to the medical bed or table 92 to further secure the positioning device 90 to the medical bed or table 92. Like discussed above, the connecting straps are preferably VELCRO® connectors.

A first medical arm and hand protection apparatus 10 is attached to the right side of the positioning device 90 and a second medical arm and hand protection apparatus 10 is attached to the left side of the positioning device 10. The apparatuses 10 are preferably sewn to the positioning device 90, but may alternatively be connected to the positioning device by fasteners or other means that would be well-known to those skilled in the art. The apparatuses 10 are preferably attached to the bottom portion 86 of the positioning device. Because the positioning device 90 is secured to the medical bed 92 (as described above) and the medical arm and hand protection apparatuses 10 are attached to the positioning device 90, a sheet 16 or clamp 80 is not needed to properly position and secure the hands and arms of the patient during surgery. The positioning device 90 and the attached arm and hand protection apparatuses 10 are preferably reusable. In an alternative embodiment, only one apparatus 10 is attached to the positioning device 90.

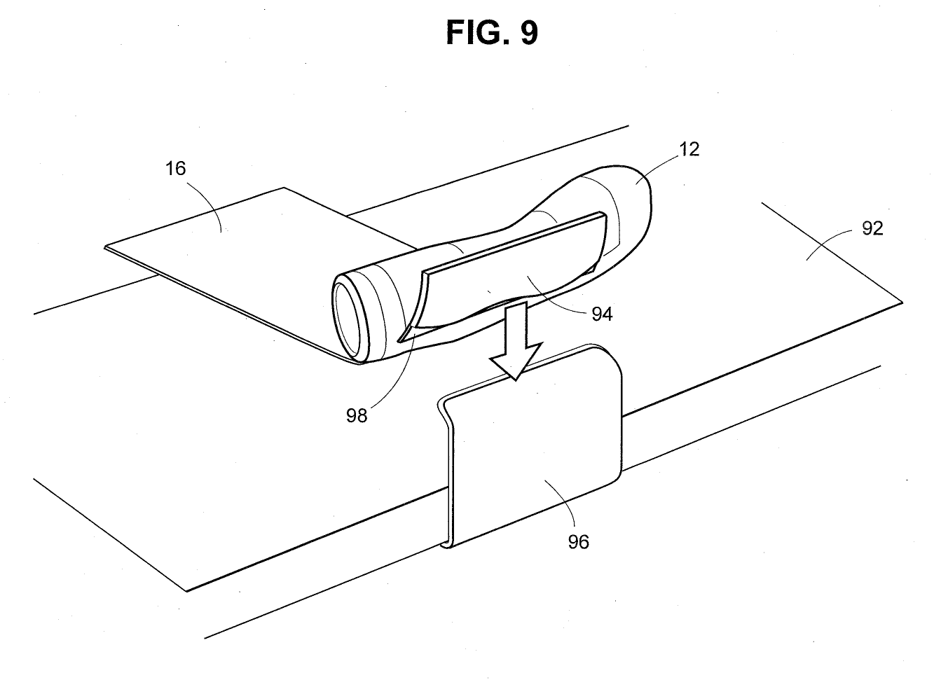

In the fifth preferred embodiment, as shown in FIG. 9, the medical hand and arm protection apparatus 10 of one of the previous embodiments also includes a slip cover 94. The slip cover 94 is attached to the protective shell 12 by means well-known to those skilled in the art, including but not limited to, gluing and stitching. The slip cover 94 preferably is attached to the bottom section 26 of the protective shell 12. The slip cover 94 includes a pocket 98 that is slid over the top of a surgical sled 96 attached to the medical bed 92. In use, the bottom portion of the surgical sled is positioned under the mattress of the medical bed or otherwise attached to the medical bed to anchor the sled in place and the top portion of the sled extends above the top surface of bed to prevent the patient's arm from sliding off the medical bed. In this regard, the surgical sled becomes a part of the medical bed. The slip cover is preferably made of stretchable material to aid in sliding the slip cover over the surgical sled. As a result of the pocket 98 of the slip cover 94 receiving the surgical sled 96, the medical hand and arm protection apparatus is secured to the surgical sled. Thus, while the sheet 16 may still be used to anchor the medical hand and arm protection apparatus close the body, it is not absolutely necessary because the slip cover 94 receiving the surgical sled serves a similar purpose. The hand and arm of the patient preferably is inserted into the shell 12 before the slip cover is slid over the surgical sled.

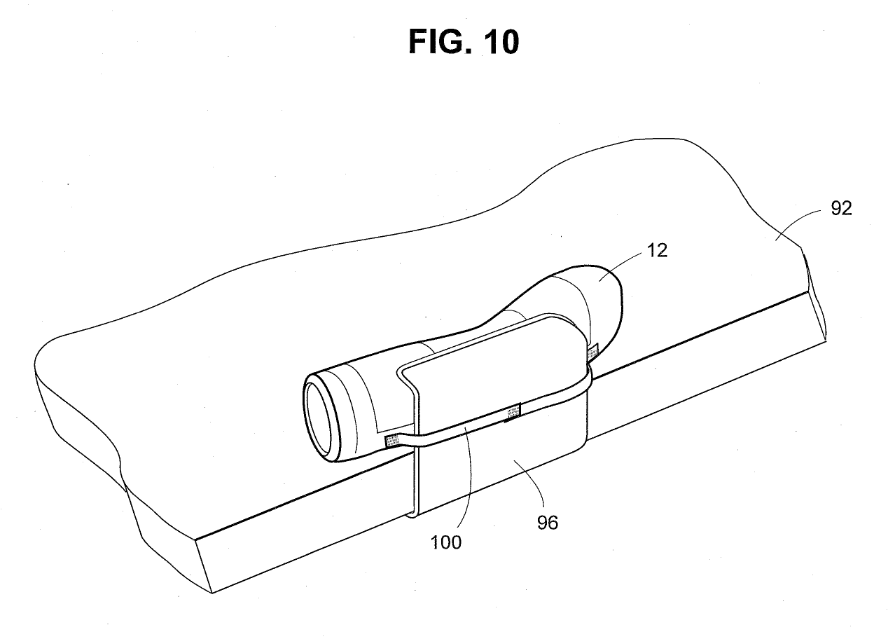

In the sixth preferred embodiment, as shown in FIG. 10, the medical hand and arm protection apparatus 10 of one of the previous embodiments also includes a sled securing strap 100. The sled securing strap 100 preferably includes two complementary connecting pieces, such as complementary VELCRO® connectors, as shown in FIG. 10. The two complementary connecting pieces are preferably positioned to wrap around the surgical sled 96 and connect to one another on the outside surface of the sled (i.e. the surface away from the patient). The sled securing strap 100 serves the same purpose as the slip cover 94 described above. While the sheet 16 may still be used to anchor the medical hand and arm protection apparatus close the body, it is not absolutely necessary because the sled securing strap serves a similar purpose. As described herein, the clamp 80, the slip cover 94, and the sled securing strap 100 may be referred to as attachment devices because each are used to attach the protective shell to a medical bed or table.

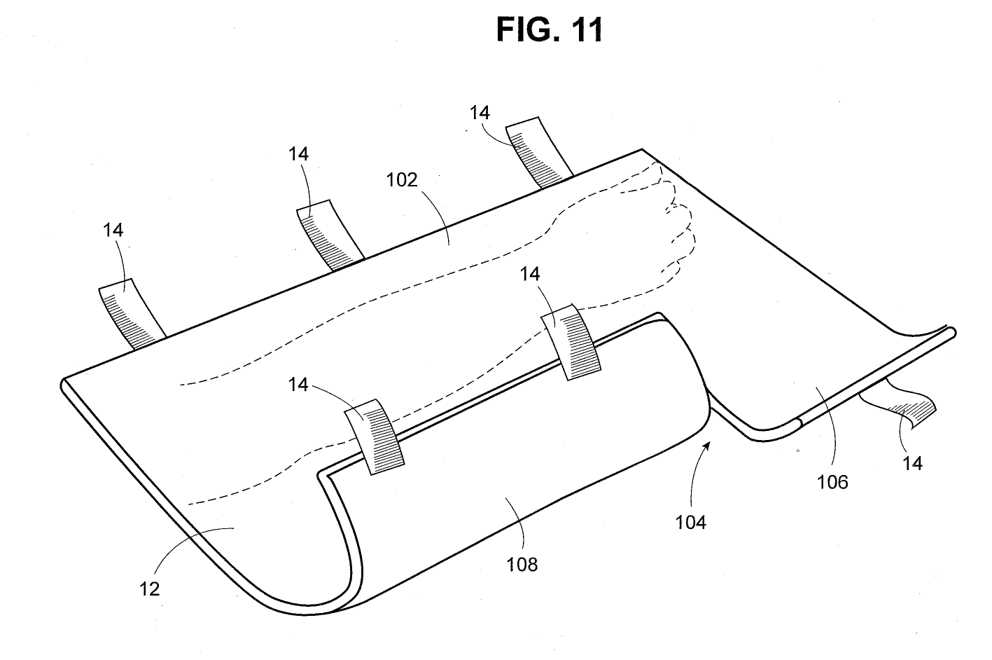

In the seventh preferred embodiment, as shown in FIG. 11, the protective shell 12 is not completely enclosed. Instead, the protective shell 12 includes a flat bottom portion 102 and a curved top portion 104 that wraps around to cover the hand and arm of the patient. The bottom portion 102 is preferably one piece. The patient's hand and arm rest on the bottom portion 102. While the end of the shell 12 is open, the patient's fingers are still protected because they are not positioned at the very end of the shell 12. The top portion 104 is joined to and folds over the bottom portion 102. The top portion 104 preferably includes two flaps 106, 108. The first flap 106 covers the hand of the patient. The second flap 108 covers the arm of the patient. Flap 106 is releasable connectable to the bottom portion 102 with a connecting strap 14. Flap 108 is releasable connectable to the bottom portion 102 with a series of connecting straps 14. Multiple connecting straps 14 are preferably used with flap 108 because of its length. The connecting straps can be undone by the medical professional when the patient's hand or arm needs to be checked. The outer surface of the foam shell 12 preferably includes a vinyl layer that is easily wipeable to sanitize the apparatus between uses.

The present invention has been described with certain preferred and alternative embodiments that are intended to be exemplary only and not limiting to the full scope of the invention.

What is claimed is:

1. A medical hand and arm protection apparatus comprising:
(a) a protective shell operable to receive a patient's arm, wherein said protective shell comprises a plurality of flaps for accessing an interior of said protective shell;
(b) a plurality of connecting straps attached to said protective shell, wherein said connecting straps are operable to close said plurality of flaps; and
(c) an attachment device, wherein said attachment device is operable to attach said protective shell to a medical bed or table.

2. The apparatus of claim 1, wherein said attachment device is a slip cover.

3. The apparatus of claim 1, wherein said attachment device is a clamp.

4. The apparatus of claim 1, wherein said attachment device is a strap.

5. The apparatus of claim 1, wherein said protective shell comprises an open end and a closed end.

6. The apparatus of claim 1, wherein said protective shell comprises two open ends.

7. The apparatus of claim 1, further comprising a positioning device.

8. The apparatus of claim 1, wherein said positioning device comprises a plurality of connecting straps operable to secure said positioning device to said medical bed or table.

9. The apparatus of claim 1, wherein said protective shell has a top section and a bottom section, wherein said top section comprises a permanent opening for viewing said interior of said protective shell.

10. The apparatus of claim 1, wherein said protective shell comprises a pocket, wherein said pocket is operable to receive a support plate.

11. The apparatus of claim 10, wherein said attachment device is connected to said support plate.

12. The apparatus of claim 1, further comprising a sheet operable for tucking under a patient's body.

13. A medical hand and arm protection apparatus comprising:
(a) a protective shell operable to receive a patient's arm, wherein said protective shell comprises a first open end and a second open end, wherein said protective shell comprises a bottom surface and a top surface, wherein said top surface comprises a first flap and a second flap;
(b) a first connecting strap configured to secure said first flap to said bottom surface of said protective shell; and
(c) a second connecting strap configured to secure said second flap to said bottom surface of said protective shell.

14. The apparatus of claim 9, wherein said protective shell is comprised of foam and vinyl.
